(19) United States
(12) Patent Application Publication
(10) Pub. No.: US 2005/0245559 A1
(43) Pub. Date: Nov. 3, 2005

(54) Treatment and prevention of prostate cancer

(76) Inventors: Hari K. Koul, Englewood, CO (US); Sweaty Koul, Englewood, CO (US)

Correspondence Address: SHERIDAN ROSS PC, 1560 BROADWAY, SUITE 1200, DENVER, CO, 80202, US

(21) Appl. No.: 11/042,644

(22) Filed: Jan. 24, 2005

Related U.S. Application Data
(60) Provisional application No. 60/538,961, filed on Jan. 23, 2004.

(51) Int. Cl. A61K 31/4745

(52) U.S. Cl. 514/283

(57) ABSTRACT

The present invention provides novel methods of preventing and treating prostate cancer with tetrandrine, a bisphehol-isoquinoline alkaloid isolated from the Chinese herb Stephania tetrandra. The present invention also provides the use of tetrandrine for the manufacture of a pharmaceutical composition for treating prostate cancer. The tetrandrine can be used as any isomer of tetrandrine such as the (R,R), (S,S), (R,S), and (S,R) isomers and any combination of these isomers. The present invention also provides methods of inhibiting the growth of prostate cancer cells in vitro by contacting the cells with tetrandrine. The present invention also provides methods of inducing apoptosis in prostate cancer cells in vitro by contacting the cells with tetrandrine.

### CROSS REFERENCE TO RELATED APPLICATIONS

This application claims the benefit of priority to U.S. Provisional Patent Application No. 60/538,961, filed Jan. 23, 2004, which is incorporated herein in its entirety by this reference.

### FIELD OF THE INVENTION

The invention relates to the fields of pharmaceuticals and oncology and provides novel methods of treating prostate cancer with isoquinoline alkaloids.

### BACKGROUND OF THE INVENTION

Prostate cancer is the second leading cause of cancer related deaths in males. Initially, prostate cancer growth and progression is dependent upon androgenic hormones. The importance of androgens in prostate cancer is demonstrated by the fact that at least 75% of prostate cancer with metastatic potential is androgen dependent at the time of diagnosis. This androgen dependence has been exploited by several therapies commonly referred to as “androgen ablation therapy.” However, these therapies have considerable side effects. Moreover, most of these therapies eventually fail and the prostate cancer progresses to androgen independence, also referred to as “hormone-refractory prostate cancer.” Most prostate cancer deaths result from emergence of this androgen resistant phenotype of prostate cancer. To date no satisfactory treatment options are available for these patients with androgen-resistant prostate cancer. Thus, there is a great need for new therapies that can prevent and treat prostate cancer in both the hormone dependent and hormone independent stages and thereby improve the outlook for patients diagnosed with prostate cancer.

### SUMMARY OF THE INVENTION

The invention provides methods of treating and preventing prostate cancer by contacting prostate cancer cells with tetrandrine. As used herein, tetrandrine refers to (1B)-6,6′,7,12-tetramethoxy-2,2′-dimethyl-berbaman and isomers thereof. Exposure of prostate cancer cells to tetrandrine causes necrosis and apoptosis of the cells in a dose- and time-dependent manner.

The tetrandrine may be a mixture of isomers of (1B)-6,6′,7,12-tetramethoxy-2,2′-dimethyl-berbaman, or a pharmaceutically-acceptable acid addition salt thereof. Alternatively, the tetrandrine may be any individual isomer of (1B)-6,6′,7,12-tetramethoxy-2,2′-dimethyl-berbaman, including the (S,S) isomer, the (R,S) isomer, the (S,R) isomer, and the (R,R) isomer.

The prostate cancer cells to be treated may be either hormone responsive or hormone refractory. Preferably, the tetrandrine is administered to achieve a concentration of between about 10 μM and about 60 μM tetrandrine in the area surrounding the cells, i.e. in the body of a mammal having prostate cancer cells, in cell culture fluid surrounding prostate cancer cells in culture, in the prostate gland, and the like.

The tetrandrine may administered as any suitable pharmaceutical composition comprising tetrandrine and pharmaceutically-acceptable excipient(s).

### BRIEF DESCRIPTION OF THE DRAWINGS

FIG. 1A shows tetrandrine inhibition of the growth and proliferation of LNCAP cells as stimulated by dihydrotestosterone (DHT) in a dose and time-dependent manner at concentrations between about 10 μM and about 60 μM.

Similarly, FIG. 1B shows tetrandrine inhibition of the growth and proliferation of LNCaP cells as stimulated by fetal calf serum (FCS) at concentrations between about 10 μM and about 60 μM.

FIG. 2A shows the effects of tetrandrine on the growth and proliferation of cultured LNCAP cells growing under the stimulus of FCS. In the top panel of FIG. 2A, LNCaP cells are shown at lower (left) and higher (right) magnification. In the middle panel of FIG. 2A, LNCaP cells treated with 10 μM tetrandrine are shown at the same lower and higher magnification showing the decrease in cell number and the notable change in cell morphology. Similarly, the bottom panel of FIG. 2A shows LNCaP cells treated with 20 μM tetrandrine at the same lower and higher magnification wherein the decrease in cell number and the change in cell morphology is even more apparent.

FIG. 2B shows a similar comparison of LNCaP cells contacted with tetrandrine in concentrations of about 10 μM to about 20 μM in the presence of FCS and DHT. These photographs show the effects on cell number and morphology of LNCaP prostate cancer cells as stimulated by DHT and the decrease in cell number and atrophied appearance of these cells caused by contact with tetrandrine while in the presence of the growth stimulant DHT.

FIG. 3 shows a summary of dose response curves for the growth and proliferation of LNCAP cells grown without (control) or in the presence of tetrandrine in concentrations between about 5 μM and about 60 μM over 24 hours. The optical density of the stained live cells is shown at each concentration of tetrandrine after 24 hours exposure.

FIG. 4 shows the effects of increasing concentrations of tetrandrine on hormone-independent PC3 prostate cancer cells after 96 hours of exposure. These results clearly show a dose-dependent killing of these hormone-independent prostate cancer cells exposed to tetrandrine.

FIG. 5 shows the inhibition of the clonogenic activity of PC3 prostate cancer cells by tetrandrine in a dose dependent manner.

FIG. 6 shows a tetrandrine concentration-dependent increase in Caspase-8, Caspase-9, Caspase-3 and Caspase-7 activation.

FIG. 7 shows a tetrandrine concentration-dependent increase in PARP cleavage.

FIG. 8 shows a tetrandrine concentration-dependent decrease in mitochondrial cytochrome-C and a concomitant tetrandrine concentration-dependent increase in cytoplasmic cytochrome-C.

FIG. 9 shows a tetrandrine concentration-dependent down-regulation of Bcl-XL in prostrate cancer cells.

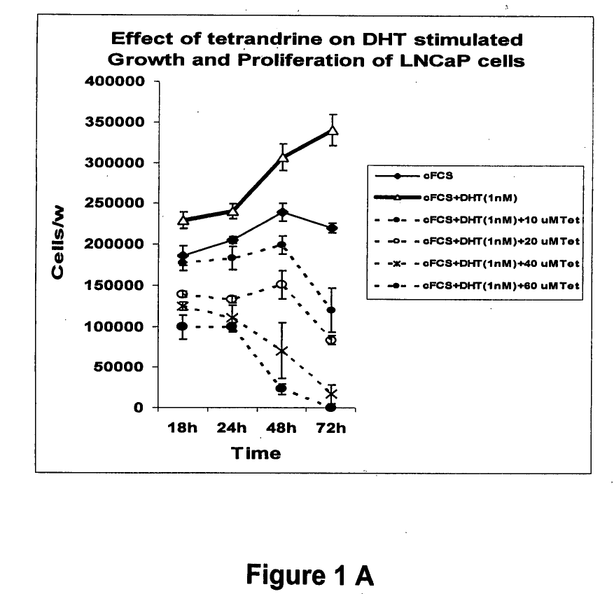

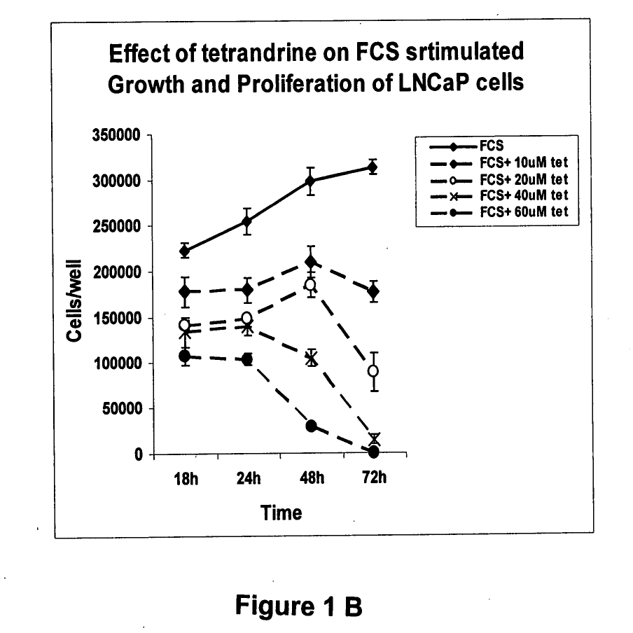

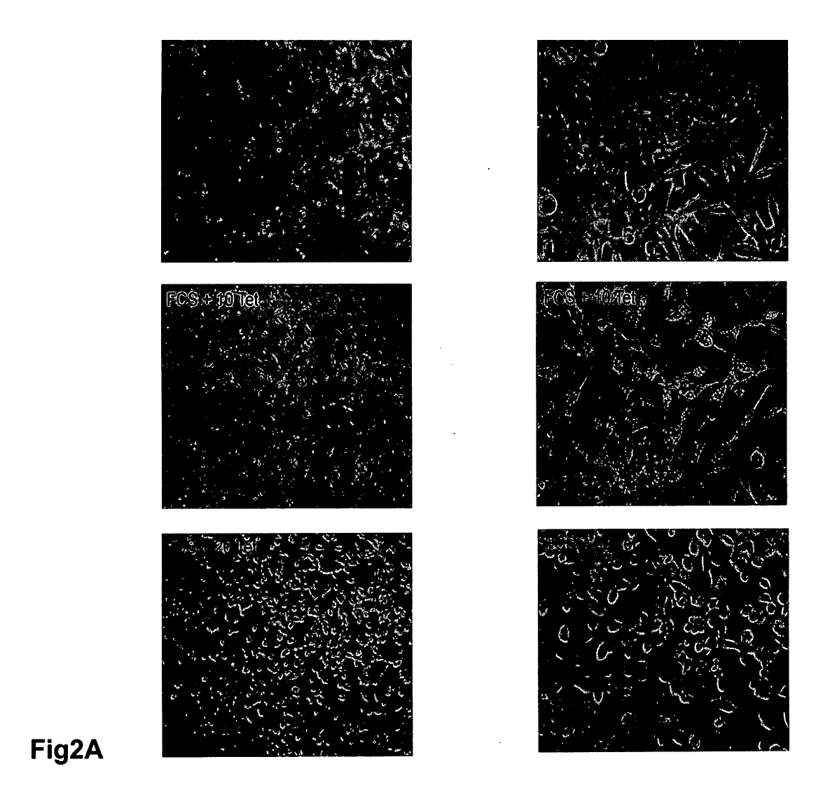

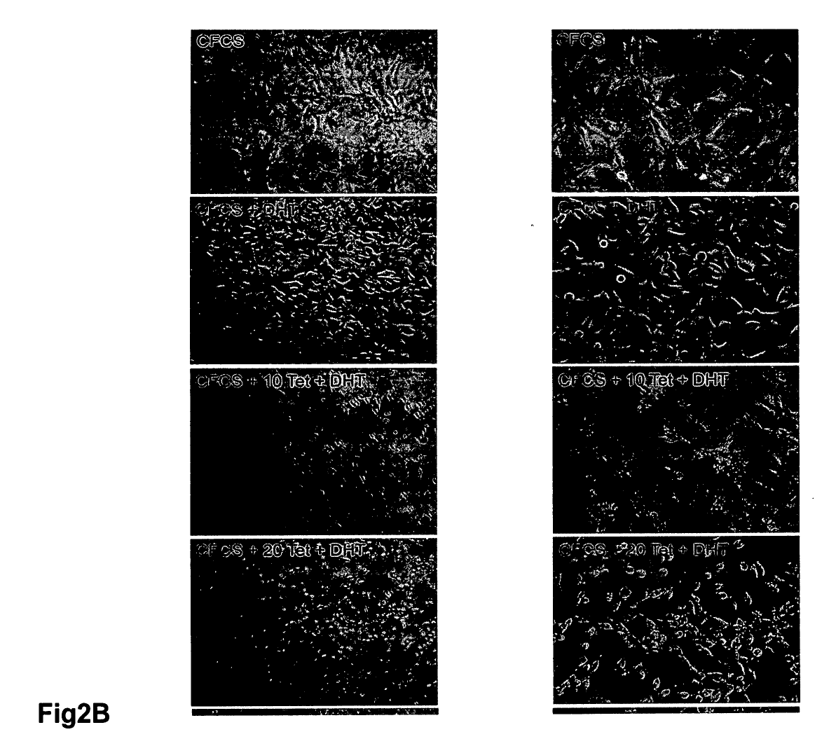

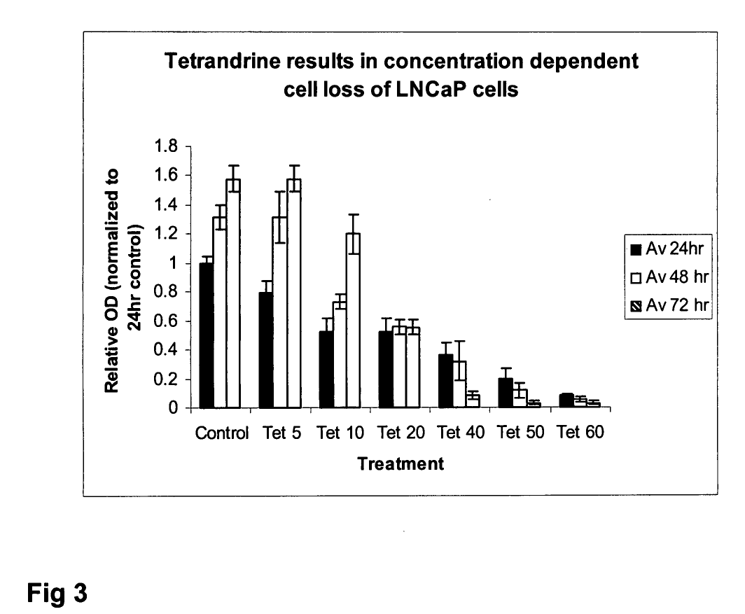

### DESCRIPTION OF THE INVENTION

The present invention provides novel methods of preventing and treating prostate cancer with tetrandrine. Tetrandrine is a bisphenol-isoquinoline alkaloid isolated from the Chinese herb Stephania tetrandra. Tetrandrine is known to exhibit a broad range of pharmacological actions including, anti-hypertensive, anti-fibrotic, anti-inflammatory, immunosuppressive, and cytoprotective properties. The mechanisms by which tetrandrine causes these diverse actions have not been clearly delineated.

The term “treating” for purposes of the present invention, includes prophylaxis or prevention, amelioration or elimination of a named condition once the condition has been established.

The term “patient” for purposes of the present invention is defined as any warm blooded animal such as, but not limited to, a mouse, guinea pig, dog, horse, or human. Preferably, the patient is human.

For purposes of the present invention, the term “prostate cancer” is defined as any malignant or neoplastic cell or cells that is, or was, originally located in the prostate of a male patient including cells that are indicative of any clinical stage of prostate cancer.

Tetrandrine is effective in treating prostate cancer as defined above. Also, tetrandrine is useful in treating neoplastic cells that have metastasized from the prostate of a male patient to reside in organs or tissues of the patient outside of the prostate gland. The effectiveness of tetrandrine in preventing and treating prostate cancer is evident from the effects of tetrandrine on LNCaP cells, a line of hormone-responsive prostate cancer cells and on PC3 cells, a line of hormone-refractory prostate cancer cells. This data show that tetrandrine is effective for arresting the growth and killing hormone-dependent and hormone-independent prostate cancer cells. Therefore, in accordance with the present invention, there is provided a method of treating prostate cancer comprising the administration to a patient in need of such treatment of an effective amount of tetrandrine.

Prior studies of tetrandrine for various other uses have indicated a minimal toxicity at doses of from 100 mg/day to 300 mg/day. Tetrandrine and several tetrandrine derivatives were screened for calcium channel blocker activity, and such was found to be minimal. Thus, the toxicity problems associated with higher doses of calcium channel blockers such as verapamil are not observed in members of the tetrandrine family.

Tetrandrine inhibits the growth and proliferation of prostate cancer cells in a dose- and time-dependent manner. For example, the growth and proliferation of LNCaP cells stimulated by dihydrotestosterone (DHT) and fetal calf serum (FCS) can be stopped or inhibited and, at higher concentrations or at longer exposure times, the cells are killed. Preferably, the concentration of tetrandrine used is between about 10 μM and about 60 μM. When LNCaP cells growing under the stimulus of FCS are exposed to tetrandrine, the cells show a sharp decrease in cell number and a significant change in cell morphology. The cells take on an atrophied appearance after contact with tetrandrine. Similarly, studies of the effects on the morphology of cellular organelles of prostrate cancer cells treated with tetrandrine showed that at concentrations of about 10 μM and 20 μM, the nuclei of prostate cancer cells appear to shrink and round while the cytoskeleton is disrupted. Tetrandrine treatment also inhibits DHT-stimulated prostate specific antigen (PSA) expression in a dose-dependent manner in LNCAP cells.

The growth and proliferation of LNCAP cells grown without (control) or in the presence of tetrandrine was studied for tetrandrine concentrations between about 5 μM and about 60 μM over 24 hours. The stained live cells showed that at concentrations of about 5 μM tetrandrine, the growth of LNCaP cells is inhibited while at a concentration of about 60 μM, almost all LNCAP cells are killed. Similarly, studies conducted using the same study methods with longer exposure times of 48 hours and 72 hours are consistent with the shorter time exposures with the exception that the death of LNCaP cells is greater at these longer exposure times at each concentration of tetrandrine.

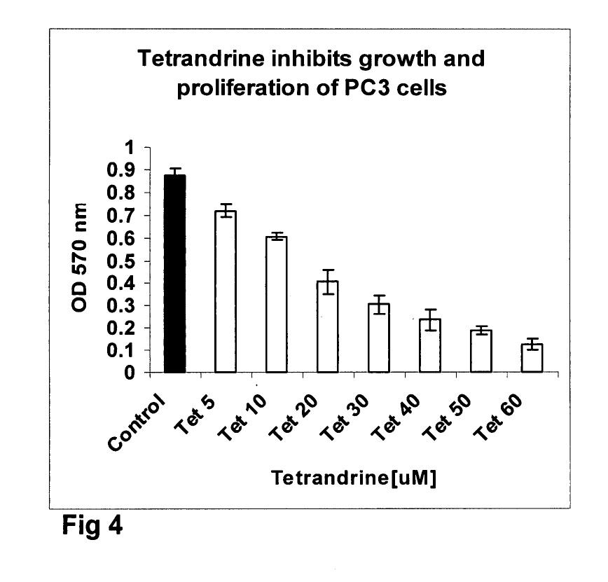

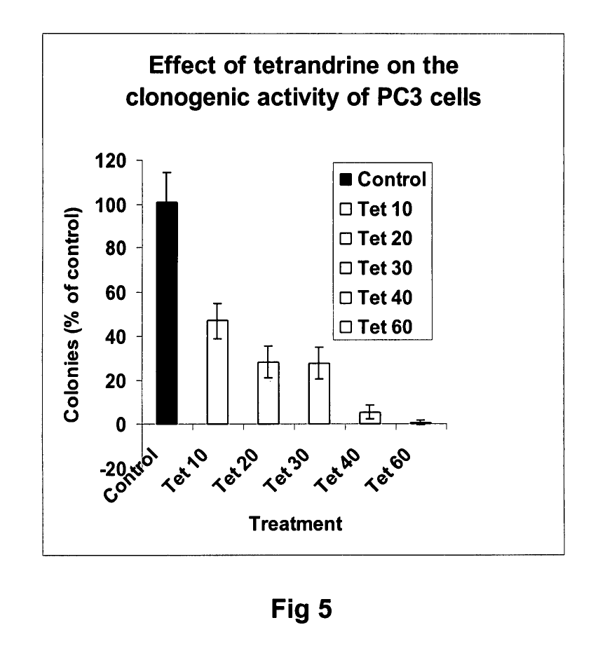

Dose response curves for the growth and proliferation of LNCaP cells grown without (control) or in the presence of tetrandrine in concentrations between about 5 μM and about 60 μM over 24 hours are shown in FIG. 4. These results are consistent with the results showing time-and dose-dependent killing of prostate cancer cells by tetrandrine.

The increasing apoptosis and necrosis in hormone-independent and hormone dependent prostate cancer cells after exposure to tetrandrine occurs in a dose- and time-dependent manner. For example, flow cytometry studies conducted on hormone-dependent LNCaP prostate cancer cells exposed to tetrandrine for 24 hours or 48 hours showed increasing apoptosis and necrosis in a dose- and time-dependent manner. Similarly, increasing concentrations of tetrandrine cause a dose-dependent killing of the hormone-independent PC3 prostate cancer cells. Without intending to be bound to any one theory, the mechanism of the dose-dependent decrease in the proliferation of prostate cancer cells appears or the dose-dependent increase in apoptosis of prostate cancer cells appears to be cell cycle arrest in G1. Tetrandrine also inhibits the clonogenic activity of prostate cancer cells in a dose-dependent manner.

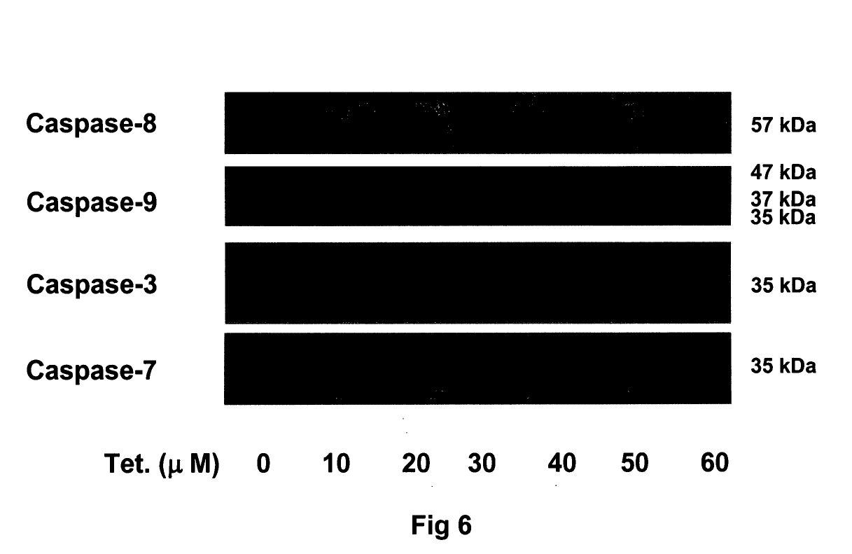

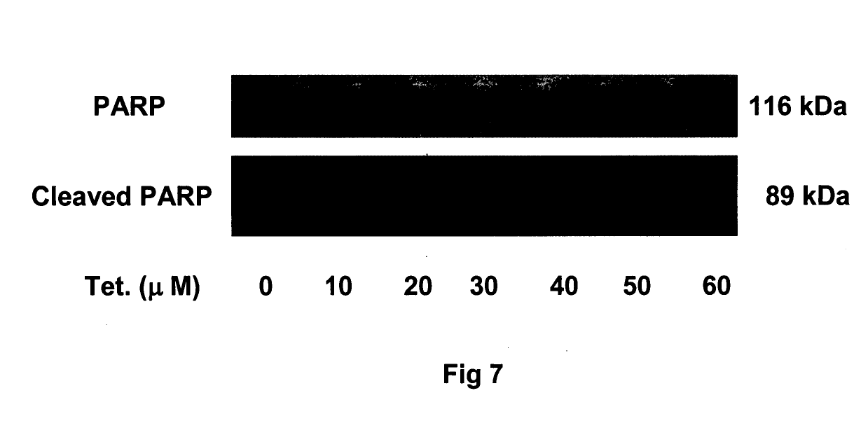

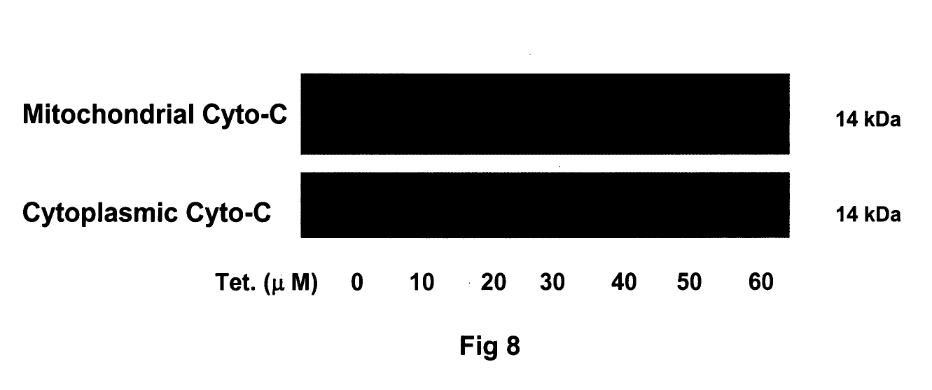

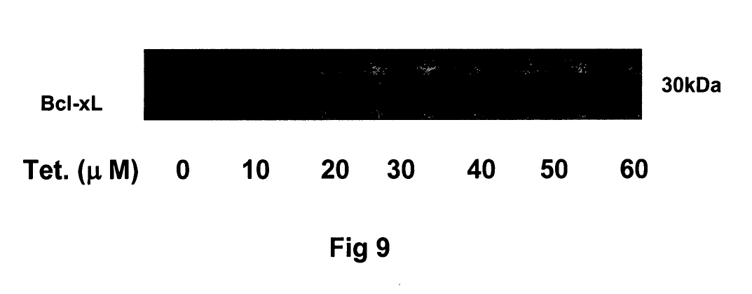

Further evidence of an apoptotic mechanism of prostate cancer cell death caused by tetrandrine includes the evidence of Caspase activation, PARP cleavage stimulation, mitochondrial cytochrome C release and down regulation of Bcl-XL shown in FIGS. 6-9.

Tetrandrine, [(1B)-6,6′,7,12-tetramethoxy-2,2′-dimethyl-berbaman], has the following chemical structure:

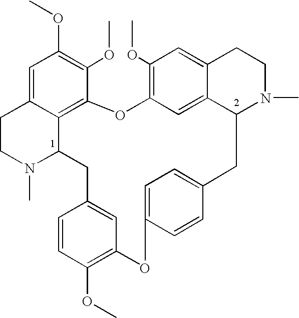

The carbons numbered 1 and 2 in the structure are chiral carbons and the configuration of these carbons may be either R or S. Accordingly, the present invention includes not only a mixture of the R and S isomers, but also the respective isolated (R,R), (S,S), (R,S), and (S,R) isomers. The present invention includes the pharmaceutically acceptable acid addition salts of tetrandrine. Since the tetrandrine compounds are amines, they are basic in nature and accordingly react with any number of inorganic and organic acids to form pharmaceutically-acceptable acid addition salts. Since the free amines of the invention are typically oils at room temperature, it is preferable to convert the free amines to their corresponding pharmaceutically-acceptable acid addition salts, which are typically solid at room temperature, for ease of handling. Acids commonly employed to form such salts include inorganic acids such as hydrochloric, hydrobromic, hydroiodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, β-hydroxybutyrate, glycollate, maleate, tartrate, methanesulfonate, propanesulfonate, naphthalene-1-sulfonate, naphthalene-2-sulfonate, mandelate and the like salts. Preferred pharmaceutically acceptable acid addition salts include those formed with mineral acids such as hydrochloric acid and hydrobromic acid, and especially those formed with organic acids such oxalic acid and maleic acid. The term “tetrandrine” as used herein, refers to any acid addition salt or the free base of any single isomer or mixture of isomers of the tetrandrine molecules. Therefore, the present invention also provides for the use of tetrandrine for the manufacture of a pharmaceutical composition for treating prostate cancer.

Tetrandrine is usually administered as a mixture of two or more isomers. Tetrandrine is available commercially from many sources such as Sigma™ and A.G. Scientific™. Tetrandrine can be purchased as the (S,S)-(+) isomer. Tetrandrine can also be isolated from the roots of Stephania Tetrandra as described in U.S. Pat. No. 6,218,541 or administered as a component of herbal medicine as described in U.S. Pat. No. 6,350,476. The tetrandrine compounds can be administered by a variety of routes including the oral, rectal, transdermal, subcutaneous, intravenous, intramuscular, intraurethral or intranasal routes. The compositions preferably are formulated in unit dosage form, meaning physically discrete units suitable as a unitary dosage, or a predetermined fraction of a unitary dose to be administered in a single or multiple dosage regimen to human subjects and other mammals, each unit containing a predetermined quantity of active material calculated to produce the desired therapeutic effect in association with a suitable pharmaceutical excipient. The compositions can be formulated so as to provide an irradiate, sustained or delayed release of active ingredient after administration to the patient by employing procedures well known in the art.

Pharmaceutical compositions thus comprise one or more compounds of the present invention associated with at least one pharmaceutically acceptable carrier, diluent or excipient. In preparing such compositions, the active ingredients are usually mixed with or diluted by an excipient or enclosed within such a carrier which can be in the form of a capsule or sachet. When the excipient serves as a diluent, it may be a solid, semi-solid, or liquid material which acts as a vehicle, carrier, or medium for the active ingredient. Thus, the compositions can be in the form of tablets, pills, powders, elixirs, suspensions, emulsions, solutions, syrups, soft and hard gelatin capsules, suppositories, sterile injectable solutions and sterile packaged powders. Examples of suitable excipients include lactose, dextrose, sucrose, sorbitol, mannitol, starch, gum acacia, calcium silicate, microcrystalline cellulose, polyvinylpyrrolidinone, cellulose, water, syrup, and methyl cellulose, the formulations can additionally include lubricating agents such as talc, magnesium stearate and mineral oil, wetting agents, emulsifying and suspending agents, preserving agents such as methyl- and propylhydroxybenzoates, sweetening agents or flavoring agents.

Tetrandrine is effective over a wide dosage range and is generally administered in an amount therapeutically effective for the treatment of prostate cancer. It will be understood, however, that the amount of the compound actually administered will be determined by a physician, in the light of the relevant circumstances, including the condition to be treated, the chosen route of administration, the actual compound administered, the age, weight, and response of the individual patient, the severity of the patient's symptoms, and the like.

The tablets or pills of the present invention may be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

1. A method for preventing or treating prostate cancer comprising administering to a patient in need of such treatment an effective amount of tetrandrine.

2. The method of claim 1, wherein the tetrandrine is (1B)-6,6′,7,12-tetramethoxy-2,2′-dimethyl-berbaman, or a pharmaceutically acceptable acid addition salt thereof.

3. The method of claim 1, wherein the tetrandrine is an isomer of (1B)-6,6′,7,12-tetramethoxy-2,2′-dimethyl-berbaman, or a pharmaceutically acceptable acid addition salt thereof selected from the group consisting of the (S,S) isomer, the (R,S) isomer, the (S,R) isomer, and the (R,R) isomer.

4. The method of claim 1, wherein the prostate cancer is hormone responsive.

5. The method of claim 1, wherein the prostate cancer is hormone refractory.

6. The method of claim 1, wherein the tetrandrine is administered in a dosing regimen to achieve a concentration in the prostate gland of between about 10 μM and about 60 μM.

7. The method of claim 1, wherein the tetrandrine is administered as a pharmaceutical composition comprising tetrandrine and at least one pharmaceutically-acceptable excipient.

8. A method of inducing apoptosis in prostate cancer cells comprising contacting the cells with tetrandrine.

9. The method of claim 8, wherein the tetrandrine is (1B)-6,6′,7,12-tetramethoxy-2,2′-dimethyl-berbaman, or a pharmaceutically acceptable acid addition salt thereof.

10. The method of claim 8, wherein the tetrandrine is an isomer of (1B)-6,6′,7,12-tetramethoxy-2,2′-dimethyl-berbaman, or a pharmaceutically acceptable acid addition salt thereof selected from the group consisting of the (S,S) isomer, the (R,S) isomer, the (S,R) isomer, and the (R,R) isomer.

11. The method of claim 20, wherein the prostate cancer cells are hormone responsive.

12. The method of claim 20, wherein the prostate cancer cells are hormone refractory.

13. The method of claim 20, wherein the cells are contacted with tetrandrine in a concentration of between about 10 μM and about 60 μM.

14. A method of inhibiting the growth of prostate cancer cells in vitro comprising contacting the cells with tetrandrine.

15. The method of claim 14, wherein the tetrandrine is (1B)-6,6′,7,12-tetramethoxy-2,2′-dimethyl-berbaman, or a pharmaceutically acceptable acid addition salt thereof.

16. The method of claim 14, wherein the tetrandrine is an isomer of (1B)-6,6′,7,12-tetramethoxy-2,2′-dimethyl-berbanan, or a pharmaceutically acceptable acid addition salt thereof selected from the group consisting of the (S,S) isomer, the (R,S) isomer, the (S,R) isomer, and the (R,R) isomer.

17. The method of claim 14, wherein the prostate cancer cells are hormone responsive.

18. The method of claim 14, wherein the prostate cancer cells are hormone refractory.

19. The method of claim 14, wherein the cells are contacted with tetrandrine in a concentration of between about 10 μM and about 60 μM.
